(19) United States
(12) Patent Application Publication
(10) Pub. No.: US 2006/0165759 A1
(43) Pub. Date: Jul. 27, 2006

(54) Antacid lozenge containing micronized particles

(75) Inventors: Atma Chaudhari, Scarborough (CA); Joseph Lee, Toronto (CA); Arthur P. G. Wright, Markham (CA); Michael Ramsay, Ajax (CA)

Correspondence Address: PFIZER, INC., 201 TABOR ROAD, MORRIS PLAINS, NJ, 07950, US

(73) Assignee: Warner-Lambert Company LLC

(21) Appl. No.: 11/044,694

(22) Filed: Jan. 27, 2005

(51) Int. Cl. A61K 9/68; A61K 33/10

(52) U.S. Cl. 424/440; 424/687

(57) ABSTRACT

An organoleptically acceptable antacid lozenge including at least one antacid present in the form of micronized particles having a particle size of less than about 10 microns. The lozenge is a solid oral composition capable of dissolving slowly in the mouth for long lasting administration of an antacid at a sustained or slow release rate. The lozenge is non-gritty, smooth textured, and does not irritate the oral mucosa.

### FIELD OF THE INVENTION

The present invention relates to a slow-dissolving confectionery composition containing one or more antacids. More particularly the invention relates to a lozenge containing micronized antacid particles.

### BACKGROUND OF THE INVENTION

Lozenges are hard candy compositions which, by their very nature, dissolve slowly in the oral cavity. Lozenges have been used extensively as effective vehicles for administering active ingredients to humans via the oral cavity. The slow dissolving property of a lozenge permits the active ingredient to be released in a controlled manner over an extended period of time. This allows the consumer to avoid taking repeated dosages at short intervals thus lowering the overall effective dosing amounts required to achieve the same therapeutic effect. The slow release of the active further minimizes overdosing that may subsequently result in adverse side effects. However, since the lozenge remains in the mouth for an extended period of time, any objectionable taste characteristics of the lozenge are magnified.

Antacid salts have been incorporated into slow dissolving compositions such as lozenges, to provide controlled delivery of antacids to the esophagus and stomach. The delivered antacid neutralizes stomach acid over a sustained time period, an effective mode for reduction of stomach acid since the gradual release of the antacid counteracts the effect of stomach emptying and the continuous secretion of acid. Additionally, the lining of the esophagus is continuously bathed, thus providing relief for tissues inflamed by gastric reflux.

Antacid salts however, do not dissolve easily in the mouth. They are frequently perceived as having unpleasant organoleptic properties such as grittiness, chalkiness, and the like. They impart to the lozenge an objectionable mouthfeel and rough texture and can adversely irritate oral mucosa, an effect which is magnified in a slow dissolving composition. The result is a product that has commercial limitations, as some consumers cannot tolerate the unpleasant mouthfeel of the product. Consequently, the ability of the consumer to retain the lozenge in the mouth for extended periods of time is adversely affected negating the positive effects of the delivery system.

Accordingly, there is a need for a solid oral composition having acceptable organoleptic properties, that is capable of dissolving slowly in the mouth for long lasting administration of an antacid at a sustained or slow release rate, that is non-gritty and smooth textured, and therefore does not irritate the oral mucosa in the mouth

### SUMMARY OF THE INVENTION

The present invention provides a lozenge comprising a confectionery base and one or more antacid actives wherein the antacid actives are present as particles having a size of less than about 10 microns. In one embodiment the antacid is selected from calcium carbonate and calcium carbonate/magnesium hydroxide mixtures. In another embodiment the lozenge has a weight of from about 2.5 to 6 grams.

The invention also provides methods of administering an antacid in a solid slow dissolving confectionery composition to a patient in need of same.

### DETAILED DESCRIPTION OF THE INVENTION

The present invention is directed to an antacid lozenge. The lozenge is a solid, oral composition, useful for orally administering an antacid component to a consumer. It is specifically formulated to be non-chewable and to remain in the oral cavity as it dissolves slowly over a period of from about 5 to 20 minutes.

The antacid component of the antacid lozenge is formed from micronized particles of the antacid. Applicants have discovered that by substantially micronizing or reducing the particle size of antacid salts, a substantial improvement in organoleptic properties, including mouthfeel and taste, of the resulting product is realized. The use of micronized particles yields a product exhibiting a dense, non-gritty and smooth texture for improved mouthfeel and palatability and which does not irritate the oral mucosa. This results in an oral delivery system that is more acceptable to the consumer, which in turn facilitates administration of the active ingredient and enhances compliance with the dosage requirements. The improved mouthfeel substantially reduces or eliminates undesirable mouth irritation typically associated with prior art slow-dissolving solid oral compositions containing gritty components.

The term “micronized particles” refers to particles having a particle size range suitable to impart a non-gritty and smooth texture to the solid oral composition in the mouth of the consumer. The average size of the micronized particles will be about 10 microns or less in size. A range from about 1 to 10 microns is contemplated with a range of about 1 to 5 microns preferred with a range of about 1 to 3 microns especially preferred. It is even more preferred that at least 50% of the micronized particles have a particle size of below about 2 microns. The micronized particles may be prepared from particles greater than 10 microns in size by micropulverization using techniques known in the art such as air jet milling.

Although the present invention is described in context of an antacid composition, it will be understood that other active ingredients which may be used in the practice of the present invention and which typically possess undesirable grittiness, chalkiness and/or rough texture in a solid oral composition can likewise be micronized in accordance with the present invention.

The antacid material is a pharmaceutically acceptable solid material which is capable of neutralizing aqueous acid and in particular gastric acid. The antacid may be a sodium, aluminum, calcium, or a magnesium acid salt, or combinations thereof. Representative examples of suitable antacids include sodium bicarbonate, sodium citrate, calcium carbonate, calcium phosphate, magnesium oxide, magnesium hydroxide, magnesium carbonate, magnesium trisilicate, aluminum carbonate, aluminum hydroxide, and the like and combinations thereof. Other suitable antacids include dihydroxy aluminum sodium carbonate, dihydroxy aluminum aminoacetate, and magnesium hydroxy aluminates. Various other co-precipitates of aluminum hydroxides or carbonates with magnesium hydroxides or carbonates, hexitols, aminoacetic acid, and the like, may be used as well as combinations of the same. It is preferred that the antacid in the present solid oral composition be a calcium salt or a combination of magnesium and calcium salts, preferably utilizing calcium carbonate and magnesium hydroxide.

The amount of the antacid incorporated into the antacid lozenge is sufficient to induce a beneficial effect of acid neutralization over a desired time period. In accordance with the present invention, the time period typically is from about 15 minutes to 60 minutes, preferably from about 30 minutes to 60 minutes. The antacid may be present in amounts of from about 10% to about 35% by weight of the composition. To provide an effective therapeutic dose, each lozenge should contain from about 5 meq to about 30 meq of acid neutralizing capacity.

The antacid lozenge may have a weight of from about 2.5 to 6 grams per dosage. For calcium carbonate/magnesium hydroxide mixtures or calcium carbonate, the antacid lozenge may be formulated to contain from about 550 to 700 mg of calcium carbonate, and about 100 to 150 mg of magnesium hydroxide or from about 650 to 850 mg of calcium carbonate alone. For example, a 3 to 4 gram antacid lozenge may contain about 110 g of magnesium hydroxide and about 550 mg of calcium carbonate or about 700 mg of calcium carbonate alone while a 4 to 5 gram lozenge may contain about 135 grams of magnesium hydroxide and about 675 mg of calcium carbonate or about 800 mg calcium carbonate alone. Smaller size lozenges can be formulated to deliver partial dosages such as one-half dose delivery where desired. It is within the skill in the art to formulate a lozenge containing an effective amount per desired dose or partial dose.

The lozenge of the present invention is a confectionery composition of the hard, boiled candy type. Hard boiled candy compositions have a hard texture and an amorphous appearance. They generally contain from about 5 to 95% of a confectionery base, a product containing a carbohydrate binder or bulking agent.

The carbohydrate binder or bulking agent used in the confectionery base of the composition may be selected from a wide variety of materials. For sugar-based compositions carbohydrates include monosaccharides, disaccharides, oligosaccharides and polysaccharides such as xylose, ribulose, glucose (dextrose), mannose, galactose, fructose (levulose), sucrose, maltose, invert sugar, partially hydrolyzed starch and corn syrup solids, and mixtures thereof and the like. For sugarless confectionary bases a sugarless bulking agent is selected. These agents include, isomalt, palatinose, polydextrose, maltodextrins, hydrogenated starch hydrolysates, hydrogenated hexoses; hydrogenated disaccharides; and mixtures thereof and the like and sugar alcohols such as sorbitol, xylitol, maltitol, mannitol, galactitol, erythritol and the like and mixtures thereof. Such carbohydrates or bulking agents are well known to those skilled in the confectionery arts. For sugar bases they generally contain a confectionery base composed of a mixture of up to about 70% sugar (sucrose) and other carbohydrate bulking agents and usually up to about 92% corn syrup.

The present composition may further contain high intensity sweeteners, sweetening agents which have a sweetness intensity substantially greater than that for sucrose, and like ingredients, which impart a desirable taste to the product. Where sugarless lozenges are prepared, the presence of a high intensity sweetener is desirable. Suitable sweeteners include, but are not limited to, saccharin and its salts, cyclamates and their salts, acesulfame and its salts, talin, monellin, steviosides, dihydrochalcone, dipeptides, polyols, amino-acid based sweeteners such as aspartame, aliatame, neotame, chlorinated sucrose derivatives (sucralose), and the like, and combinations thereof. High intensity sweeteners are generally used in the range of from about 0.1% to 2% by weight of the solid oral composition.

Suitable flavorings for the antacid lozenges of this invention include both synthetic flavoring liquids and/or liquids derived from plants, leaves, flowers, fruits and so forth, as well as combinations thereof. More specific examples of suitable flavorings include mints such as spearmint, peppermint (menthol), wintergreen (methylsalicylate) and the like; fruit flavors such as citrus including lemon, orange, lime and grapefruit; and fruit essences including apple, pear, peach, cherry, grape, plum, pineapple, banana, and berry including strawberry, raspberry, blueberry and the like, and cinnamon; clove, bay, anise, eucalyptus, thyme, cedar leaf, nutmeg, allspice, sage, bitter almonds, cassia, vanilla and the like. Fruit and mint flavors are preferred in the antacid lozenge. Cooling agents such as menthol, N-ethyl-p-menthane-3-carboxamide, 3-1-menthoxy propane 1,2-diol, and the like and combinations thereof, may also be used to provide a cooling sensation.

The amount of flavoring employed is normally a matter of preference subject to such factors as flavor type, individual flavor, and strength desired. Thus, the amount may be varied in order to obtain the result desired in the final product. Such variations are within the capabilities of those skilled in the art without the need for undue experimentation. The flavorings are generally utilized in amounts that will vary depending upon the individual flavor, and may, for example, range in amounts of about 0.01% to about 3% by weight of the final composition weight.

The antacid lozenge may further have incorporated therein effective amounts of pharmaceutically acceptable additives in order to impart thereto additional desirable properties, such as, but not limited to, gelling agents, humectants, lubricants, colorants, preservatives and the like may be employed for their commonly known intended purposes. The amount of the additives may vary with the corresponding antacid selected. The selection of such pharmaceutically acceptable additives and amounts thereof to be employed is well within the skill of the art. The additives may be present in amounts of from about 0.1% to 25% by weight of the solid oral composition.

The antacid lozenges of the present invention may contain other active ingredients. Examples include, but are not limited to. Histamine₂ Receptor Antagonists such as famotidine, ranitidine, cimetidine and the like; Proton Pump Inhibitors such as omeprazole, lansoprazole and the like; alginates; bismuth subsalicylate; pectin; minerals, vitamins, fibers, and the like; breath freshening agents such as chlorophyll, menthol and the like; anti-gas agents such as simethicone and the like, and anti-cariogenic agents such as fluorides, amorphous calcium phosphate-casein phosphopeptide, xylitol and the like and combinations thereof.

The antacid lozenges of the present invention may be prepared by conventional methods established for hard boiled candy compositions in the confectionery art.

Hard, boiled candy compositions may be routinely prepared by conventional batch methods such as those involving fire cookers, vacuum cookers, and scraped-surface cookers also referred to as high speed atmospheric cookers, or they may be prepared by extrusion methods using twin or single screw extruders.

In the batch method, boiled candy lozenges are made by first mixing at least the carbohydrate binder or bulking agent in a stainless steel vessel to about 140° C. The mixture is heated until most of the moisture is driven off. The mixture is allowed to cool somewhat, and the remaining ingredients may be mixed into the batch. In the practice of the present invention it is preferred to include the antacid at this stage in the process. Flavorants are usually added last.

In the extrusion method the mixing and heating occur in the first sections of the barrel of the extruder at elevated temperatures with addition of additives occurring later in the extrusion process.

During the cooling process, after evaporation of moisture, the mass changes form through the liquid phase to plastic and solid. The final moisture content of the antacid lozenges is generally from about 1.5% to 3.0% by weight of the composition for a solids content of from 97% to 98.5%.

Once the candy mass has been properly tempered, it may be cut into workable portions or formed into desired shapes. A variety of forming techniques may be utilized depending upon the shape and size of the final product desired, the most common being flat, circular, rectangular, oval octagonal and biconvex forms. A general discussion of the composition and preparation of hard confections may be found in E. B. Jackson, Ed. “Sugar Confectionery Manufacture”, 2nd edition, Blackie Academic & Professional Press, Glasgow UK, (1990), at pages 129-169.

### EXAMPLES

The following examples are merely exemplary embodiments of the present invention, provided to more specifically teach and better define the compositions of the present invention. They are for illustrative purposes only. One skilled in the art will readily recognize from the above discussion, and from the accompanying claims, that various changes, modifications and variations can be made therein without departing from the spirit and scope of the invention as defined in the claims that follow.

### Examples 1-2

### Sugar Lozenges

Sugar based antacid lozenges were prepared according to the constituents in Table 1 below. The amounts are given in percent by weight.

TABLE 1

| Ingredient (percent by weight) | Example 1 | Example 2 |
|---|---|---|
| Base | | |
| Sugar | 42.92 | 42.92 |
| Corn Syrup | 42.92 | 42.92 |
| Water | 14.16 | 14.16 |
| | 100.00 | 100.00 |
| Lozenge | | |
| Base | 80.37 | 79.22 |
| Calcium Carbonate, micronized | 13.80 | 17.75 |
| Magnesium hydroxide, micronized | 2.80 | — |
| Glycerine | 2.00 | 2.00 |
| Berry flavor | 0.50 | 0.50 |
| Cooling agent WS3 | 0.01 | 0.01 |
| Sodium saccharin | 0.20 | 0.20 |
| Color | 0.32 | 0.32 |
| | 100.00 | 100.00 |

The lozenges of Table 1 were prepared by the batch method. The Example 1 lozenge was formed into 4 gram pieces each delivering 550 mg calcium carbonate and 110 mg of magnesium hydroxide. The Example 2 lozenge was formed into 4 gram pieces each delivering 710 mg calcium carbonate.

### Examples 3-5

### Sugarless Lozenges

Sugarless antacid lozenges were prepared according to the constituents in Table 2 below. The amounts are given in percent by weight.

TABLE 2

| Ingredient (percent by weight) | Example 3 | Example 4 | Example 5 |
|---|---|---|---|
| Base | | | |
| Isomalt | 73.08 | 73.08 | 73.08 |
| Lycasin* | 5.00 | 5.00 | 5.00 |
| Water | 21.92 | 21.92 | 21.92 |
| | 100.00 | 100.00 | 100.00 |
| Lozenge | | | |
| Base | 77.08 | 77.08 | 77.08 |
| Calcium Carbonate, micronized | 18.00 | 18.00 | 18.00 |
| 5% Pectin solution | 2.20 | 1.20 | 2.00 |
| Cottonseed Oil | 2.00 | 1.00 | 2.00 |
| Cherry flavor | 0.18 | — | — |
| Berry Flavor | — | 2.15 | — |
| Vanilla/Peppermint flavor | — | — | 0.40 |
| Cooling agent | 0.10 | 0.10 | 0.10 |
| Xylitol | — | 0.03 | 0.03 |
| Acesulfame K | 0.03 | 0.02 | 0.02 |
| Sucralose | 0.03 | 0.03 | 0.03 |
| Color | 0.40 | 0.40 | 0.35 |
| | 100.00 | 100.00 | 100.00 |

*(Roquette; hydrogenated starch hydrolysate)

The lozenges of Table 2 were prepared by the batch method. All three Example lozenges were formed into both a 4.5 gram piece each delivering 810 mg calcium carbonate and a 3.8 gram piece each delivering 684 mg of calcium carbonate.

What is claimed is:

1. A lozenge comprising a confectionery base and one or more antacid actives wherein said active or actives have a particle size of less than about 10 microns.

2. The lozenge of claim 1 wherein said particle size is from about 1 to about 10 microns.

3. The lozenge of claim 1 wherein said antacid is selected from a group consisting of sodium antacid salts, aluminum acid salts, calcium antacid salts, magnesium acid salts, and combinations thereof.

4. The lozenge of claim 3 wherein the antacid is selected from calcium carbonate and calcium carbonate/magnesium hydroxide mixtures.

5. The lozenge of claim 1 formulated to dissolve in the mouth of a consumer over a time period of from about 15 to about 60 minutes.

6. The lozenge of claim 1 having a weight of from about 2.5 to about 6 grams.

7. A method of administering an antacid in a solid slow dissolving confectionery composition, said method comprising administering to a patient a composition of claim 1.
